(19) United States
(12) Patent Application Publication
(10) Pub. No.: US 2021/0330296 A1
(43) Pub. Date: Oct. 28, 2021

(54) METHODS AND APPARATUSES FOR ENHANCING ULTRASOUND DATA

(71) Applicants: Nathan Silberman, Brooklyn, NY (US); Igor Lovchinsky, New York, NY (US); Swaminathan Sankaranarayanan, Guilford, CT (US); Yang Liu, Hoboken, NJ (US)

(72) Inventors: Nathan Silberman, Brooklyn, NY (US); Igor Lovchinsky, New York, NY (US); Swaminathan Sankaranarayanan, Guilford, CT (US); Yang Liu, Hoboken, NJ (US)

(73) Assignee: Butterfly Network, Inc., Guilford, CT (US)

(21) Appl. No.: 17/240,994

(22) Filed: Apr. 26, 2021

Related U.S. Application Data
(60) Provisional application No. 63/016,243, filed on Apr. 27, 2020.

(51) Int. Cl. A61B 8/08; A61B 8/00; G06N 3/08

(52) U.S. Cl. CPC A61B 8/5269; A61B 8/0883; G06N 3/08; A61B 8/466; A61B 8/469; A61B 8/0866

(57) ABSTRACT

Aspects of the technology described herein relate to enhancing ultrasound data. Some embodiments include receiving ultrasound data and automatically determining, with a processing device, that the ultrasound data depicts an anatomical view or one of a set of anatomical views. Based on automatically determining that the ultrasound data depicts the anatomical view or one of the set of anatomical views, an option is enabled to perform ultrasound data enhancement specific to the anatomical view or the set of anatomical views. Based on receiving the selection of the option, the ultrasound data, a portion thereof, and/or subsequently-collected ultrasound data is enhanced using the ultrasound data enhancement specific to the anatomical view or the set of anatomical views.

### CROSS-REFERENCE TO RELATED APPLICATIONS

This application claims the benefit of U.S. Provisional Application Ser. No. 63/016,243 filed on Apr. 27, 2020, and entitled “METHODS AND APPARATUSES FOR ENHANCING ULTRASOUND DATA,” which is hereby incorporated by reference herein in its entirety.

### FIELD

Generally, the aspects of the technology described herein relate to ultrasound data. Some aspects relate to enhancing ultrasound data.

### BACKGROUND

Ultrasound probes may be used to perform diagnostic imaging and/or treatment, using sound waves with frequencies that are higher than those audible to humans. Ultrasound imaging may be used to see internal soft tissue body structures. When pulses of ultrasound are transmitted into tissue, sound waves of different amplitudes may be reflected back towards the probe at different tissue interfaces. These reflected sound waves may then be recorded and displayed as an image to the operator. The strength (amplitude) of the sound signal and the time it takes for the wave to travel through the body may provide information used to produce the ultrasound image. Many different types of images can be formed using ultrasound devices. For example, images can be generated that show two-dimensional cross-sections of tissue, blood flow, motion of tissue over time, the location of blood, the presence of specific molecules, the stiffness of tissue, or the anatomy of a three-dimensional region.

### SUMMARY

According to one aspect of the application, an apparatus includes a processing device configured to receive ultrasound data; automatically determine that the ultrasound data depicts an anatomical view or one of a set of anatomical views; based on automatically determining that the ultrasound data depicts the anatomical view or one of the set of anatomical views, enable an option to perform ultrasound data enhancement specific to the anatomical view or the set of anatomical views; receive a selection of the option; and based on receiving the selection of the option, enhance the ultrasound data, a portion thereof, and/or subsequently-collected ultrasound data using the ultrasound data enhancement specific to the anatomical view or the set of anatomical views.

According to another aspect of the application, an apparatus includes a processing device configured to receive ultrasound data; automatically determine that the ultrasound data depicts an anatomical view or one of a set of anatomical views; and based on automatically determining that the ultrasound data depicts the anatomical view or one of the set of anatomical views, enhance the ultrasound data, a portion thereof, and/or subsequently-collected ultrasound data using the ultrasound data enhancement specific to the anatomical view or the set of anatomical views.

According to another aspect of the application, an apparatus includes a processing device configured to receive ultrasound data; automatically determine that the ultrasound data does not depict an anatomical view or one of a set of anatomical views specific to ultrasound data enhancement being performed; based on automatically determining that the ultrasound data does not depict the anatomical view or one of the set of anatomical views, enable an option to cease to perform the ultrasound data enhancement specific to the anatomical view or the set of anatomical views; receive a selection of the option; and based on receiving the selection of the option, cease to enhance the ultrasound data, a portion thereof, and/or subsequently-collected ultrasound data using the ultrasound data enhancement specific to the anatomical view or the set of anatomical views.

According to another aspect of the application, an apparatus includes a processing device configured to receive ultrasound data; automatically determine that the ultrasound data does not depict an anatomical view or one of a set of anatomical views specific to ultrasound data enhancement being performed; and based on automatically determining that the ultrasound data does not depict the anatomical view or one of the set of anatomical views, cease to enhance the ultrasound data, a portion thereof, and/or subsequently-collected ultrasound data using the ultrasound data enhancement specific to the anatomical view or the set of anatomical views.

In some embodiments, the processing device is in operative communication with an ultrasound device, and the processing device is configured, when receiving the ultrasound data, to receive the ultrasound data from the ultrasound device in real-time as the ultrasound data is collected or generated by the ultrasound device. In some embodiments, the processing device is configured, when receiving the ultrasound data, to retrieve ultrasound data that has been previously stored.

In some embodiments, the processing device is configured, when automatically determining that the ultrasound data depicts or does not depict the anatomical view or one of the set of anatomical views, to use one or more statistical models and/or deep learning techniques.

In some embodiments, the anatomical view comprises one of an apical two chamber view of a heart, an apical four chamber view of the heart, a parasternal long axis view of the heart, and a parasternal short axis view of the heart. In some embodiments, the set of anatomical views comprises an apical two chamber view of a heart, an apical four chamber view of the heart, a parasternal long axis view of the heart, and a parasternal short axis view of the heart. In some embodiments, the anatomical view comprises a three-dimensional view of a fetus.

In some embodiments, the processing device is configured, when enabling the option, to enable a user to select the option. In some embodiments, the processing device is configured, when enabling the option, to enable an action to be performed upon selection of the option. In some embodiments, the processing device is configured, when enabling the option, to display an option that was not previously displayed by the processing device. In some embodiments, the processing device is configured, when enabling the option, to change a manner of display of the option.

In some embodiments, the processing device is configured, when enhancing the ultrasound data, the portion thereof, and/or the subsequently-collected ultrasound data, to use a statistical model trained to convert ultrasound data from an initial domain to a final domain, where the initial domain includes low-quality ultrasound data and the final domain includes high-quality ultrasound data. In some embodiments, the statistical model is specifically trained on ultrasound data depicting the anatomical view or the set of anatomical views. In some embodiments, the processing device is configured, when enhancing the ultrasound data, the portion thereof, and/or the subsequently-collected ultrasound data, to enhance the ultrasound data using a user-selectable degree of enhancement. In some embodiments, the processing device is configured, when enhancing the ultrasound data, the portion thereof, and/or the subsequently-collected ultrasound data, to enhance portions of the ultrasound data non-uniformly. In some embodiments, the processing device is configured, when enhancing the portions of the ultrasound data non-uniformly, to enhance first portions of an ultrasound image more than second portions of the ultrasound image, the first portions being closer to a user-selected point than the second portions. In some embodiments, the processing device is configured, when enhancing the ultrasound data, the portion thereof, and/or the subsequently-collected ultrasound data, to only enhance a portion of the ultrasound data. In some embodiments, the processing device is configured, when only enhancing the portion of the ultrasound data, to only enhance a portion of an ultrasound image within a user-selectable region.

### BRIEF DESCRIPTION OF THE DRAWINGS

Various aspects and embodiments will be described with reference to the following exemplary and non-limiting figures. It should be appreciated that the figures are not necessarily drawn to scale. Items appearing in multiple figures are indicated by the same or a similar reference number in all the figures in which they appear.

FIG. 1A is a flow diagram illustrating an example process for enhancing ultrasound images, in accordance with certain embodiments described herein;

FIG. 1B is a flow diagram illustrating an example process for enhancing ultrasound images, in accordance with certain embodiments described herein;

FIG. 1C is a flow diagram illustrating an example process for enhancing ultrasound images, in accordance with certain embodiments described herein;

FIG. 1D is a flow diagram illustrating an example process for enhancing ultrasound images, in accordance with certain embodiments described herein;

FIG. 2 illustrates an example graphic user interface (GUIs) that may be displayed by a processing device in an ultrasound system, in accordance with certain embodiments described herein;

FIG. 3 illustrates an example graphic user interface (GUIs) that may be displayed by a processing device in an ultrasound system, in accordance with certain embodiments described herein;

FIG. 4 illustrates an example graphic user interface (GUIs) that may be displayed by a processing device in an ultrasound system, in accordance with certain embodiments described herein;

FIG. 5 illustrates an example graphic user interface (GUIs) that may be displayed by a processing device in an ultrasound system, in accordance with certain embodiments described herein;

FIG. 6 illustrates an example graphic user interface (GUIs) that may be displayed by a processing device in an ultrasound system, in accordance with certain embodiments described herein;

FIG. 7 illustrates an example graphic user interface (GUIs) that may be displayed by a processing device in an ultrasound system, in accordance with certain embodiments described herein;

FIG. 8 illustrates an example graphic user interface (GUIs) that may be displayed by a processing device in an ultrasound system, in accordance with certain embodiments described herein;

FIG. 9 illustrates an example graphic user interface (GUIs) that may be displayed by a processing device in an ultrasound system, in accordance with certain embodiments described herein; and

FIG. 10 illustrates a schematic block diagram of an example ultrasound system upon which various aspects of the technology described herein may be practiced.

### DETAILED DESCRIPTION

Statistical models may be used for enhancing images, such as ultrasound images, or more generally, ultrasound data. In some embodiments, a statistical model may be trained to convert ultrasound data from an initial domain to a final domain, where the initial domain includes low-quality ultrasound data and the final domain includes high-quality ultrasound data. For example, the initial domain may include ultrasound data collected by an ultrasound device that collects lower quality ultrasound data and the final domain may include ultrasound data collected by an ultrasound device that collects higher quality ultrasound data.

The inventors have recognized that such a statistical model may be specifically trained on ultrasound data depicting a particular anatomical view or a particular set of anatomical views. This may mean that the statistical model may only operate to enhance ultrasound data when the inputted ultrasound data depicts the same view as or one of the same views as the ultrasound data on which the statistical model was trained. If ultrasound data depicting one anatomical view is inputted to a statistical model specifically trained to enhance ultrasound data depicting another anatomical view, the output ultrasound data may be worse in quality than the original. Thus, the inventors have developed technology that, in some embodiments, enables the option to enhance ultrasound data only upon a determination that the ultrasound data depicts the particular anatomical view or one of the particular set of anatomical views on which the enhancement statistical model has been trained. In some embodiments, a statistical model (e.g., different than the enhancement statistical model) may be used to automatically determine whether ultrasound data depicts the particular anatomical view or one of the particular set of anatomical views on which the enhancement statistical model has been trained, and then enable or not enable the enhancement option based on this automatic determination. As one example, an ultrasound system may have a cardiac image enhancement feature (e.g., using a statistical model trained on cardiac ultrasound images) installed. If the ultrasound system detects that an ultrasound image displayed by the ultrasound system depicts a cardiac view, the system may enable an option for the user to select to enhance the ultrasound image using the cardiac enhancement feature. In some embodiments, upon a determination that the ultrasound data depicts the particular anatomical view or one of the particular set of anatomical views on which the enhancement statistical model has been trained, enhancement may be automatically performed, without requiring the user to select an option to perform the enhancement.

In some embodiments, the processing device may perform enhancement specific to a particular anatomical view or a set of anatomical views without determining that the ultrasound image depicts the particular anatomical view or one of the set of anatomical views. As one example, a user ultrasound system may have a cardiac image enhancement feature (e.g., using a statistical model trained on cardiac ultrasound images) installed. If the user selects a cardiac preset (i.e., a set of imaging parameter values), image enhancement specific to cardiac views may be automatically performed. An option to cease to enhance ultrasound data may be enabled upon a determination that the ultrasound data does not depict the particular anatomical view or one of the particular set of anatomical views on which the enhancement statistical model has been trained.

Various aspects of the present disclosure may be used alone, in combination, or in a variety of arrangements, and is therefore not limited in its application to the details and arrangement of components set forth in the foregoing description or illustrated in the drawings. For example, aspects described in one embodiment may be combined in any manner with aspects described in other embodiments.

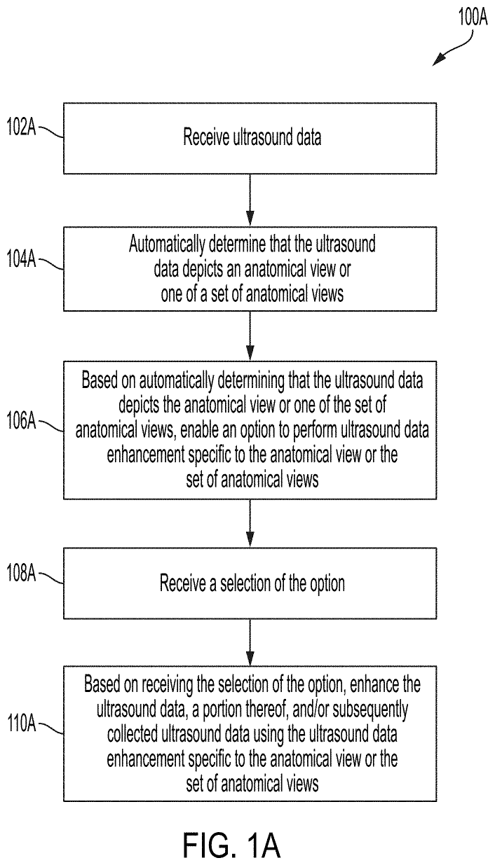

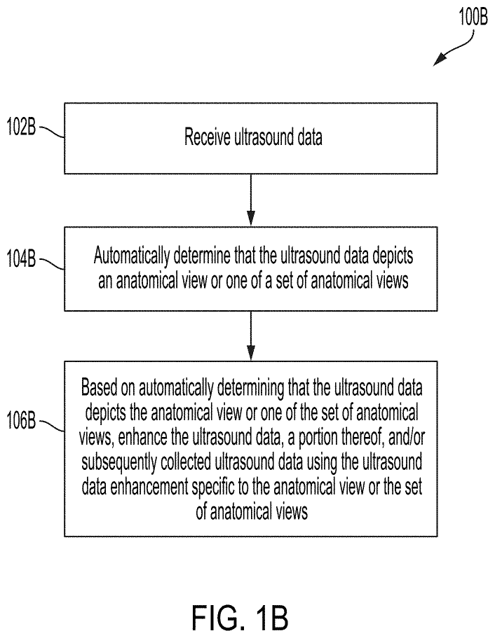

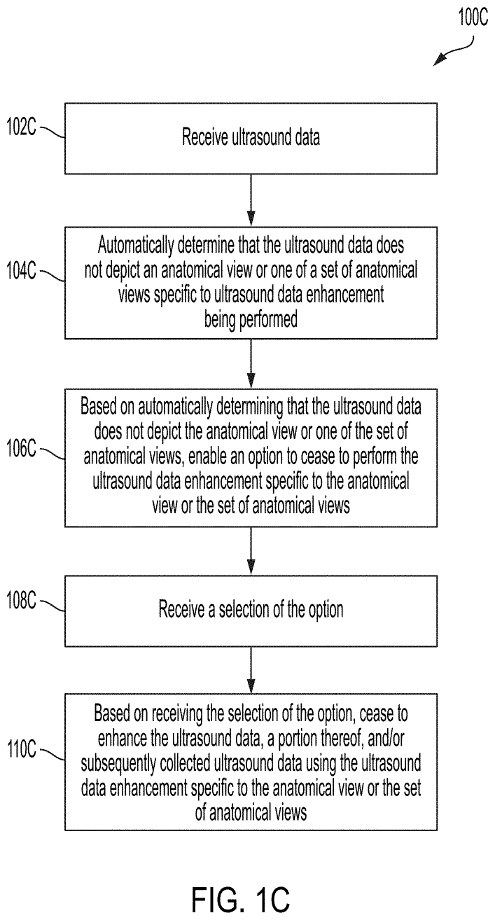

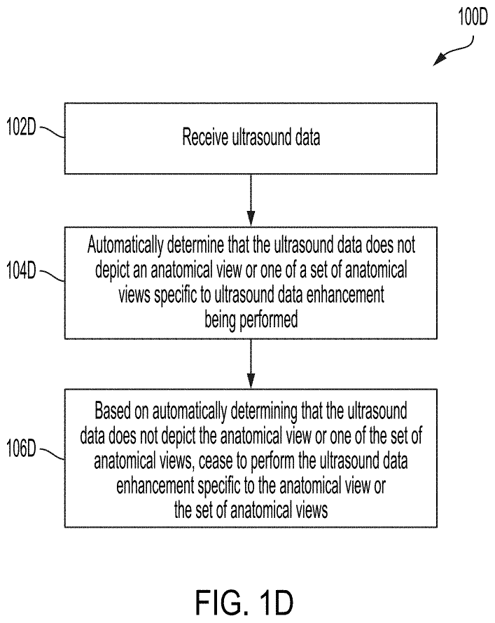

FIGS. 1A-1D are flow diagrams illustrating example processes 100A-100D, respectively, for enhancing ultrasound images, in accordance with certain embodiments described herein. The processes 100A-100D may be performed by a processing device, such as a mobile phone, tablet, or laptop. The processing device may be part of or in operative communication with an ultrasound device. The ultrasound device and the processing device may communicate over a wired communication link (e.g., over Ethernet, a Universal Serial Bus (USB) cable or a Lightning cable) or over a wireless communication link (e.g., over a BLUETOOTH, WiFi, or ZIGBEE wireless communication link).

Referring to FIG. 1A, in act 102A of the process 100A, the processing device receives ultrasound data. For example, the ultrasound data may be raw acoustical data, scan lines generated from raw acoustical data, and/or one or more ultrasound images (e.g., a cine) generated from raw acoustical data or scan lines. In some embodiments, the processing device may receive the ultrasound data from the ultrasound device in real-time (e.g., as the ultrasound data is collected or generated by the ultrasound device). In some embodiments, the processing device may retrieve ultrasound data that has been previously stored. For example, the processing device may receive the ultrasound data from an external electronic device such as a server or from the processing device's own internal memory. The process 100A proceeds from act 102A to act 104A.

In act 104A, the processing device automatically determines that the ultrasound data depicts an anatomical view or one of a set of anatomical views. For example, the processing device may automatically determine that one or more two-dimensional or three-dimensional ultrasound images (the ultrasound data received in act 102A or generated based on the ultrasound data received in act 102A in this example) depict a particular anatomical view. As another example, the processing device may automatically determine that one or more two-dimensional or three-dimensional ultrasound images (the ultrasound data received in act 102A or generated based on the ultrasound data received in act 102A in this example) depict one of a particular set of anatomical views. As an example of an anatomical view, in some embodiments, the processing device may determine that the ultrasound data depicts a particular standard anatomical view of the heart (e.g., that the ultrasound data depicts the apical two chamber view of the heart, that the ultrasound data depicts the apical four chamber view of the heart, that the ultrasound data depicts the parasternal long axis view of the heart, or that the ultrasound data depicts the parasternal short axis view of the heart). As an example of a set of anatomical views, in some embodiments, the processing device may determine that the ultrasound data depicts any of the standard anatomical views of the heart (e.g., that the ultrasound data depicts one of the apical two chamber view of the heart, the apical four chamber view of the heart, the parasternal long axis view of the heart, or the parasternal short axis view of the heart). As another example of an anatomical view, in some embodiments, the processing device may determine that the ultrasound data depicts a three-dimensional view of a fetus.

In some embodiments, the processing device may use one or more statistical models and/or deep learning techniques for this automatic determination. The statistical models may include a convolutional neural network, a fully connected neural network, a recurrent neural network (e.g., a long short-term memory (LSTM) recurrent neural network), a random forest, a support vector machine, a linear classifier, and/or any other statistical model. The statistical models may be trained to determine, based on ultrasound data, an anatomical view depicted by the ultrasound data. The statistical models may be trained on multiple sets of ultrasound data each labeled with the anatomical view depicted by the ultrasound data. In some embodiments, the statistical model may be stored on the processing device. In some embodiments, the processing device may access the statistical model on an external electronic device (e.g., a server). The process 100A proceeds from act 104A to act 106A.

In act 106A, based on automatically determining that the ultrasound data (e.g., an ultrasound image) depicts the anatomical view or one of a set of anatomical views, the processing device may enable an option to perform ultrasound data enhancement specific to the anatomical view or the set of anatomical views. Further description of ultrasound data enhancement may be found with reference to act 110A. Enabling the option may include enabling a user to select the option and/or enabling an action to be performed upon selection of the option. In some embodiments, the option may be a button displayed on a GUI displayed by the processing device. In some embodiments, enabling the option may include displaying the option on a GUI displayed by the processing device, where the option was not displayed previous to the determination in act 104. In some embodiments, enabling the option may include changing a manner of display of the option on a GUI displayed by the processing device (e.g., changing a color of the option or highlighting the option). In embodiments in which the option was displayed prior to enabling the option, prior to the processing device enabling the option, selection of the option (e.g., touching the option on a touch-sensitive display screen) by a user may have not caused an action to be performed. In some embodiments, enabling the option may not include any change in a display.

As one example, if the ultrasound data is an ultrasound image received in real-time, and the processing device determines that the ultrasound image depicts the anatomical view or one of a set of anatomical views, the processing device may enable the option for all subsequent ultrasound images received in real-time during the ultrasound imaging session, or for subsequent ultrasound images received in real-time for a predetermined time period afterwards. As another example, if the ultrasound data is a stored cine, and the processing device determines that at least one ultrasound image in the cine depicts the anatomical view or one of a set of anatomical views, the processing device may enable the option for other ultrasound images in the cine. As another example, if the ultrasound data is a stored cine, and the processing device determines that some but not all of the ultrasound images in the cine depict the anatomical view or one of a set of anatomical views, the processing device may only enable the option for those ultrasound images in the cine. The process 100A proceeds from act 106A to act 108A.

In act 108A, the processing device receives a selection of the option. For example, when the option is displayed on a touch-sensitive display screen of the processing device, the processing device may detect that the user has touched the option on the touch-sensitive display screen. As another example, the processing device may detect that the user has clicked the option with a mouse. As another example, the processing device may detect, through a speaker on the processing device, that the user has provided a voice command to select the option. The process 100A proceeds from act 108A to act 110A.

In act 110A, based on receiving the selection of the option, the processing device enhances the ultrasound data, a portion thereof, and/or subsequently-collected ultrasound data using the ultrasound data enhancement specific to the anatomical view or the set of anatomical views. In some embodiments, the ultrasound data enhancement may include inputting the ultrasound data to a statistical model that outputs an enhanced version of the ultrasound data. The statistical model may be trained to convert ultrasound data from an initial domain to a final domain, where the initial domain includes low-quality ultrasound data and the final domain includes high-quality ultrasound data. For example, the initial domain may include ultrasound data collected by an ultrasound device that collects lower quality ultrasound data and the final domain may include ultrasound data collected by an ultrasound device that collects higher quality ultrasound data. Quality of an ultrasound image may be based, for example, on the sharpness of the ultrasound image and the haze artifacts present in the ultrasound image. Example statistical model techniques for converting data from one domain to another include pix2pix and CycleGAN. Further description of these techniques may be found in Isola, Phillip, et al. “Image-to-image translation with conditional adversarial networks,” Proceedings of the IEEE conference on computer vision and pattern recognition, 2017, and Zhu, Jun-Yan, et al, “Unpaired image-to-image translation using cycle-consistent adversarial networks,” Proceedings of the IEEE international conference on computer vision, 2017, the contents of which are incorporated by reference herein in their entireties.

The ultrasound data enhancement is specific to the anatomical view or the set of anatomical views that the processing device determined in act 104A is depicted by the ultrasound data. In particular, the statistical model may be specifically trained on ultrasound data depicting the anatomical view or the set of anatomical views, but not others. This may mean that the statistical model may only operate to enhance ultrasound data when the inputted ultrasound data depicts the same view as or one of the same views as the ultrasound data on which the statistical model was trained. Thus, as described above with reference to acts 104A and 106A, the processing device may only provide the option to enhance ultrasound data with a statistical model trained on an anatomical view or a set of anatomical views when the ultrasound data to be inputted to the statistical model depicts the anatomical view or one of a set of anatomical views.

In embodiments in which the ultrasound data enhancement is performed by a statistical model, the processing device may input the ultrasound data received in act 102A to the statistical model. In some embodiments, the statistical model may be stored on the processing device. In some embodiments, the processing device may access the statistical model on an external electronic device (e.g., a server).

As one example, if the processing device receives the selection of the option when ultrasound data is being collected in real-time, the processing device may enhance subsequent ultrasound images received in real-time during the ultrasound imaging session, or the processing device may enhance subsequent ultrasound images received in real-time for a predetermined time period afterwards. As another example, if the processing device receives the selection of the option when displaying a stored cine, the processing device may enhance all the ultrasound images in the cine. As another example, if the processing device receives the selection of the option when displaying a stored cine, the processing device may only enhance ultrasound images in the cine that are displayed or selected when the processing device received the selection of the option.

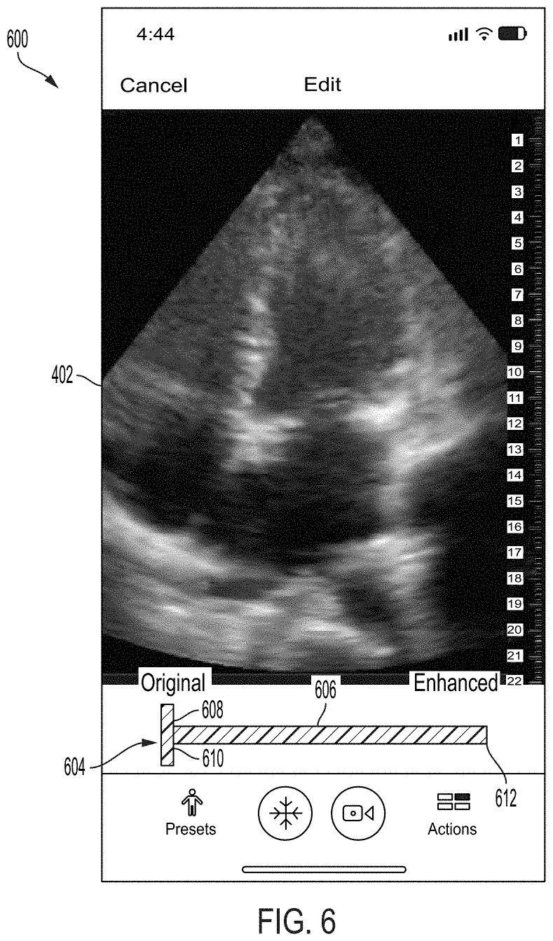

In some embodiments, if the ultrasound data is an ultrasound image, the processing device may enhance the whole ultrasound image. In some embodiments, the processing device may not fully enhance the ultrasound data, but may enhance the ultrasound data using a user-selectable degree of enhancement (e.g., chosen using a slider such as that described with reference to FIGS. 6-8). In particular, consider that f is the user-selectable degree of enhancement between 0 and 1. Further, consider that the value of a pixel at a particular location (x,y) in an original ultrasound image is original(x,y) and the value of a pixel at the particular location (x,y) in the fully enhanced ultrasound image (i.e., when any of the enhancement methods described above are applied fully and uniformly to each pixel in the ultrasound image) is enhanced(x,y). The processing device may display a final enhanced ultrasound image at act 110A where the value of each pixel is (1−f)(original(x,y))+f (enhanced(x,y)).

In some embodiments, the processing device may enhance portions of the ultrasound data non-uniformly. For example, the processing device may enhance portions of an ultrasound image that are near a user-selected point more than portions of the ultrasound image that are far from the user-selected point. In particular, the value of a given pixel in the final enhanced ultrasound image may be a weighted sum of the value of the corresponding pixel in the original ultrasound image and the value of the corresponding pixel in the ultrasound image if it were fully enhanced (i.e., when any of the enhancement methods described above are applied fully and uniformly to each pixel in the ultrasound image). For pixels closer to the selected point, the value of the pixel in the fully enhanced ultrasound image may be weighted more. For pixels farther from the selected point, the value of the pixel in the original ultrasound image may be weighted more. Weighting of the original and fully enhanced ultrasound images may therefore be location-dependent. Consider that the value of a pixel at a particular location (x,y) in the original ultrasound image is original(x,y) and the value of a pixel at the particular location (x,y) in the fully enhanced ultrasound image (generated as described above) is enhanced(x,y). Then the value of that pixel in the final ultrasound image displayed by the processing device at act 110 may be equal to (1−f(x,y))(original (x,y))+(f (x,y))(enhanced(x,y)), where f is a value between 0 and 1 determining location-dependent weighting of the pixels in the original and fully enhanced ultrasound images. In some embodiments, the location-dependent weighting may be based on a Gaussian function of the distance of the pixel at (x,y) from the location of the selected point (x0, y0). Let the distance between (x,y) and (x0,y0) be d=sqrt((x0−x)\hat{ }2+(y0−y)\hat{ }2). Then, in some embodiments, f=exp(−0.5*(d/s)\hat{ }2)/(s*sqrt(2*pi)), where s may be a hyper-parameter (e.g., chosen a-priori via visual testing) that controls the degree at which the location-dependent weighting of the fully enhanced ultrasound image versus the original ultrasound image falls off moving away from the selected point.

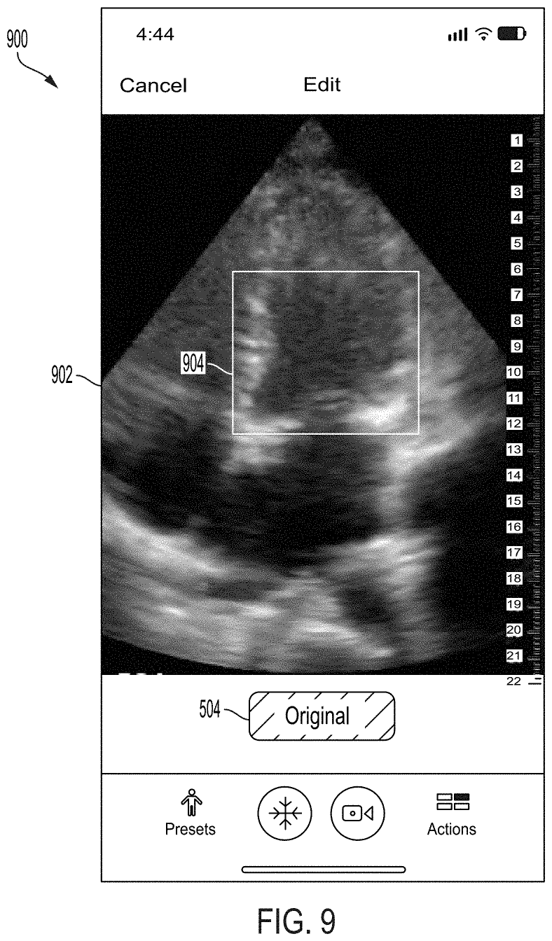

In some embodiments, the processing device may only enhance a portion of the ultrasound data. In some embodiments, the processing device may only enhance a portion of an ultrasound image within a user-selectable region (e.g., a box or other shape that a user may move across the ultrasound image, such as that described with reference to FIG. 9). In particular, consider that the value of a pixel at a particular location (x,y) in the original ultrasound image is original(x,y). Consider that, when the original ultrasound image is fully enhanced (i.e., any of the enhancement methods described above are applied fully and uniformly to each pixel in the ultrasound image), the value of a pixel at the particular location (x,y) in the enhanced ultrasound image is enhanced(x,y). Further, consider that mask is a matrix equal in size to the original and enhanced ultrasound images, where pixels in mask corresponding to the location of the user-selectable region are equal to 1 and other pixels are 0. Then the final enhanced ultrasound image displayed by the processing device at act 110A may be equal to original(x,y)*(1−mask(x,y))+enhanced(x,y)*mask(x,y), where the multiplication operator indicates pixel-by-pixel multiplication.

Referring to FIG. 1B, acts 102B and 104B are the same as acts 102A and 104A, respectively. In act 106B, based on automatically determining (at act 104B) that the ultrasound data depicts the anatomical view or one of the set of anatomical views, the processing device enhances the ultrasound data, a portion thereof, and/or subsequently collected ultrasound data using the ultrasound data enhancement specific to the anatomical view or the set of anatomical views. Further description of such enhancement may be found with reference to act 110A. Thus, in 106C, the processing device automatically performs enhancement specific to the anatomical view or one of the set of anatomical views, without requiring user selection of an option (e.g., as in the process 100A), when the processing device determines that the ultrasound data depicts the anatomical view or one of the set of anatomical views. In some embodiments, a user may select a setting that causes the processing device to automatically perform enhancement specific to the anatomical view or one of the set of anatomical views when the processing device determines that the ultrasound data depicts the anatomical view or one of the set of anatomical views. For example, the default setting may be that the user must select an option to perform enhancement (e.g., as in the process 100A), but a user may select a setting for automatic enhancement (e.g., as in the process 100B).

Referring to FIG. 1C, act 102C is the same as 102A. In the process 100C, the processing device is already performing ultrasound data enhancement (e.g., as described with reference to act 110A, and on the ultrasound data received in act 102C) specific to a particular anatomical view or set of anatomical views, without the processing device determining that the ultrasound data depicts the anatomical view or one of the set of anatomical views (e.g., as in the processes 100A and 100B). Performing ultrasound data enhancement may be a default setting, or the user may have set a setting to perform ultrasound data enhancement specific to a particular anatomical view or set of anatomical views, without the processing device determining that the ultrasound data depicts the anatomical view or one of the set of anatomical views. The processing device may perform the process 100C when, for example, the user has selected a preset (i.e., a set of imaging parameter values) specific to the anatomical view or set of anatomical views that is also specific to the ultrasound data enhancement. For example, if the user selects a cardiac preset, the processing device may by default perform ultrasound data enhancement specific to a cardiac view or views.

In act 104C, the processing device automatically determines that the ultrasound data does not depict an anatomical view or one of a set of anatomical views specific to ultrasound data enhancement being performed. In some embodiments, the processing device may use one or more statistical models and/or deep learning techniques for this automatic determination. The statistical models may include a convolutional neural network, a fully connected neural network, a recurrent neural network (e.g., a long short-term memory (LSTM) recurrent neural network), a random forest, a support vector machine, a linear classifier, and/or any other statistical model. In some embodiments, the statistical models may be trained to determine, based on ultrasound data, an anatomical view depicted by the ultrasound data. The statistical models may be trained on multiple sets of ultrasound data each labeled with the anatomical view depicted by the ultrasound data. In some embodiments, the statistical model may be stored on the processing device. In some embodiments, the processing device may access the statistical model on an external electronic device (e.g., a server). The processing device may then determine whether the anatomical view depicted by the ultrasound data matches the particular anatomical view or one of the particular set of anatomical views that is specific to the ultrasound data enhancement being performed. For example, if the processing device is performing ultrasound data enhancement specific to a cardiac view or views, and the processing device then determines that the ultrasound data received in act 102C depicts an abdominal view, the processing device may determine that the ultrasound data does not depict the anatomical view or set of anatomical views specific to the ultrasound data enhancement being performed. The process 100C proceeds from act 104C to act 106C.

In act 106C, based on automatically determining that the ultrasound data does not depict the anatomical view or one of the set of anatomical views, the processing device enables an option to cease to perform ultrasound data enhancement specific to the anatomical view or the set of anatomical views. Act 106C is the same as the act 106A, except that the option is to cease to perform ultrasound data enhancement, rather than an option to perform ultrasound data enhancement. The process 100C proceeds from act 106C to act 108C.

In act 108C, the processing device receives a selection of the option. Act 108C is the same as the act 108A, except that the option is to cease to perform ultrasound data enhancement, rather than an option to perform ultrasound data enhancement.

In act 110C, based on receiving the selection of the option, the processing device ceases to enhance the ultrasound data, a portion thereof, and/or subsequently collected ultrasound data using the ultrasound data enhancement specific to the anatomical view or the set of anatomical views.

Referring to FIG. 1D, acts 102D and 104D are the same as acts 102B and 104B, respectively. In act 106D, based on automatically determining (at act 106D) that the ultrasound data does not depict the anatomical view or one of the set of anatomical views specific to the ultrasound data enhancement being performed, the processing device ceases to enhance the ultrasound data, a portion thereof, and/or subsequently collected ultrasound data using the ultrasound data enhancement specific to the anatomical view or the set of anatomical views. Thus, in 106D, the processing device automatically ceases to perform enhancement specific to the anatomical view or one of the set of anatomical views, without requiring user selection of an option (e.g., as in the process 100B), when the processing device determines that the ultrasound data does not depict the anatomical view or one of the set of anatomical views.

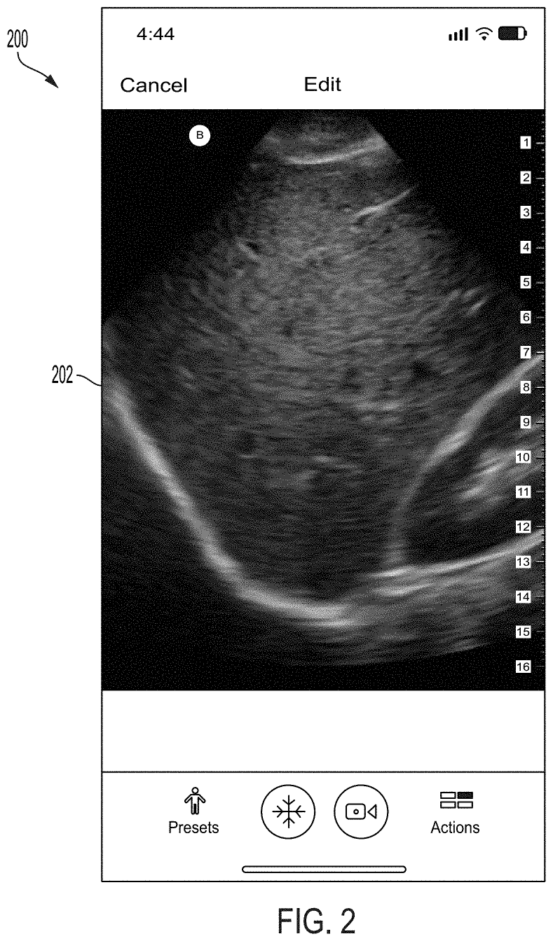

FIGS. 2-9 illustrate example graphic user interfaces (GUIs) that may be displayed by a processing device in an ultrasound system, in accordance with certain embodiments described herein. The processing device may be, for example, a mobile phone, tablet, or laptop. The processing device may be in operative communication with an ultrasound device, and the ultrasound device and the processing device may communicate over a wired communication link (e.g., over Ethernet, a Universal Serial Bus (USB) cable or a Lightning cable) or over a wireless communication link (e.g., over a BLUETOOTH, WiFi, or ZIGBEE wireless communication link). In some embodiments, the ultrasound device itself may display the GUIs. It should be appreciated that the forms of the GUIs illustrated in the figures are non-limiting, and other GUIs performing the same functions with different forms may also be used.

FIG. 2 illustrates an example GUI 200. The GUI 200 includes an ultrasound image 202.

In some embodiments, the ultrasound image 202 may be displayed in real-time as ultrasound imaging is being performed. For example, the ultrasound device may have collected ultrasound data and transmitted the ultrasound data to the processing device, and the processing device may have generated the ultrasound image from the ultrasound data and displayed the ultrasound image. As another example, the ultrasound device may have generated the ultrasound image based on the ultrasound data and transmitted the ultrasound image to the processing device, and the processing device may have displayed the ultrasound image. In some embodiments, the ultrasound image 202 may have been previously stored to memory, and the processing device may have retrieved the ultrasound image 202 from the memory. For example, the processing device may have retrieved the ultrasound image 202 from a temporary storage buffer on the processing device, from permanent memory on the processing device, or from an external device (e.g., a server). In some embodiments, the ultrasound image 202 may be part of a cine that was previously stored to memory, and the processing device may have retrieved the cine from the memory and displayed ultrasound images in the cine one after another.

The processing device may determine (e.g., using a statistical model), that the ultrasound image 202 depicts a particular anatomical view or one of a particular set of anatomical views. For example, the processing device may determine that the ultrasound image 202 depicts Morison's pouch, or that the ultrasound image 202 depicts one of a particular set of abdominal views (e.g., including the view of Morison's pouch). In the GUI 200, the processing device does not enable, and therefore does not display, an option for enhancing the ultrasound image 202. This may be because the processing device does not have access to an image enhancement statistical model trained on ultrasound images depicting Morison's pouch or an image enhancement statistical model trained on ultrasound images depicting a particular set of abdominal views (e.g., including the view of Morison's pouch).

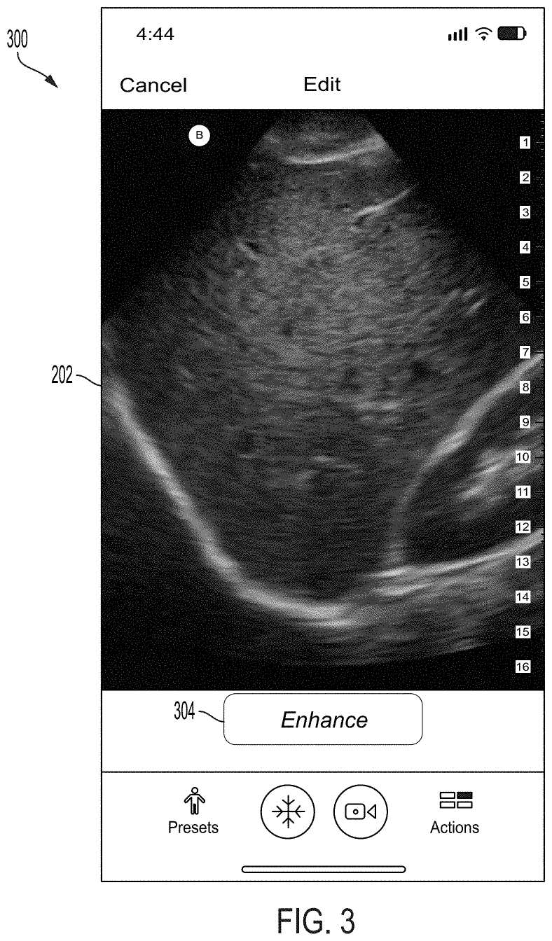

FIG. 3 illustrates an example GUI 300. The GUI 300 may be an alternative to the GUI 200. The GUI 300 includes the ultrasound image 202 and an enhancement option 304. While the processing device displays the enhancement option 304, the enhancement option 304 is not enabled. In other words, if a user tries to select the enhancement option 304 (e.g., by touching the enhancement option 304 on a touch-sensitive display screen), no image enhancement may be performed. In the example of FIG. 3, the enhancement option 304 may also be displayed with a format indicating that the enhancement option 304 is not enabled. As in the GUI 200, the enhancement option 304 may not be enabled because the processing device does not have access to an image enhancement statistical model trained on ultrasound images depicting Morison's pouch or an image enhancement statistical model trained on ultrasound images depicting a particular set of abdominal views (e.g., including the view of Morison's pouch).

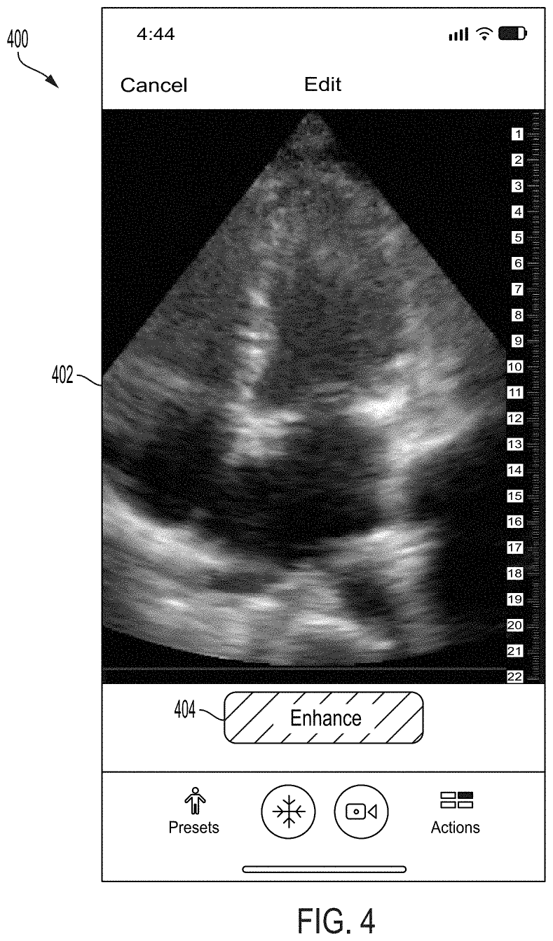

FIG. 4 illustrates an example GUI 400. The GUI 400 includes an ultrasound image 402 and an enhancement option 404. In some embodiments (e.g., when the processing device is implementing the process 100A), the processing device may display the GUI 400 after displaying the GUI 200 or the GUI 300. In some embodiments, the ultrasound image 202 may have been collected and the processing device may have displayed the ultrasound image 202 in real-time, and then the ultrasound image 402 may have been collected and the processing device may have displayed the ultrasound image 402 in real-time after the ultrasound image 202 was displayed. In some embodiments, the ultrasound image 202 and the ultrasound image 402 (or cines containing the ultrasound images) may have been previously stored, and the processing device may have retrieved and displayed the ultrasound image 202 and then retrieved and displayed the ultrasound image 402 (or retrieved and displayed the cines one after another).

The processing device may determine (e.g., using a statistical model), that the ultrasound image 402 depicts a particular anatomical view or one of a particular set of anatomical views. For example, the processing device may determine that the ultrasound image 402 depicts the apical four-chamber view of the heart, or that the ultrasound image 202 depicts one of a particular set of cardiac views (e.g., the four standard cardiac views, including the apical four-chamber view). The processing device displays and enables the enhancement option 404. This may be because the processing device has access to an image enhancement statistical model trained on ultrasound images depicting the apical four-chamber view of the heart or an image enhancement statistical model trained on ultrasound images depicting a particular set of cardiac views (e.g., the four standard cardiac views, including the apical four-chamber view). If a user selects the enhancement option 404 (e.g., by touching the enhancement option 404 on a touch-sensitive display screen), image enhancement may be performed. Additionally, if the processing device displays the GUI 400 after the GUI 300, the enhancement option 404 may be displayed with a format (e.g., different than the format of the enhancement option 304) indicating that the enhancement option 404 is enabled.

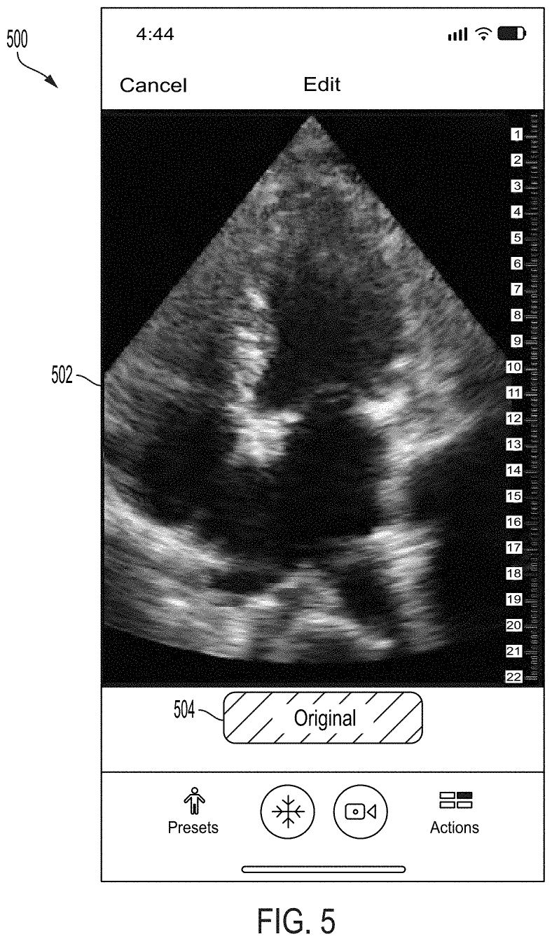

FIG. 5 illustrates an example GUI 500. The GUI 500 includes an ultrasound image 500 and an original option 504. In some embodiments (e.g., when the processing device is implementing the process 100A), the processing device may display the GUI 500 after receiving a selection of the enhancement option 404 from the GUI 400. The ultrasound image 502 may be an enhanced version of the ultrasound image 402. In some embodiments, the processing device may generate the ultrasound image 502 by inputting the ultrasound image 402 to a statistical model that outputs an enhanced version of the ultrasound data. The statistical model may be trained to convert ultrasound data from an initial domain to a final domain, where the initial domain includes low-quality ultrasound data and the final domain includes high-quality ultrasound data. For example, the initial domain may include ultrasound data collected by an ultrasound device that collects lower quality ultrasound data and the final domain may include ultrasound data collected by an ultrasound device that collects higher quality ultrasound data. Example statistical model techniques for converting data from one domain to another include pix2pix and CycleGAN. Further description of these techniques may be found in Isola, Phillip, et al. “Image-to-image translation with conditional adversarial networks,” Proceedings of the IEEE conference on computer vision and pattern recognition, 2017, and Zhu, Jun-Yan, et al, “Unpaired image-to-image translation using cycle-consistent adversarial networks,” Proceedings of the IEEE international conference on computer vision, 2017. The statistical model may be specifically trained on ultrasound images depicting the anatomical view depicted by the ultrasound image 402 or on a set of anatomical views including the view depicted by the ultrasound image 402. If the ultrasound image 402 was displayed in real-time, the processing device may continue to enhance subsequent ultrasound images collected by the ultrasound device in real-time, or may continue to enhance subsequent ultrasound image collected by the ultrasound device in real-time for a predetermined time period. If the ultrasound image 402 was previously stored, the processing device may continue to enhance subsequent ultrasound images that are retrieved. If the ultrasound image 402 is displayed as part of a previously stored cine, the processing device may enhance all the ultrasound images in the cine while displaying the cine, or may only enhance certain ultrasound images in the cine (e.g., only the ultrasound image 502 currently displayed, or only ultrasound images in the cine that depict the anatomical view or set of anatomical views on which the statistical model is trained).

In some embodiments, in response to receiving a selection of the original option 504, the processing device may display the GUI 400 (i.e., show the original ultrasound image 402 rather than the enhanced ultrasound image 502). If the ultrasound 402 was displayed in real-time, the processing device may cease to enhance subsequent ultrasound images collected by the ultrasound device in real-time. If the ultrasound image 402 was previously stored, the processing device may cease to enhance subsequent ultrasound images that are retrieved. If the ultrasound image 402 is displayed as part of a previously stored cine, the processing device may cease to enhance ultrasound images in the cine while displaying the cine.

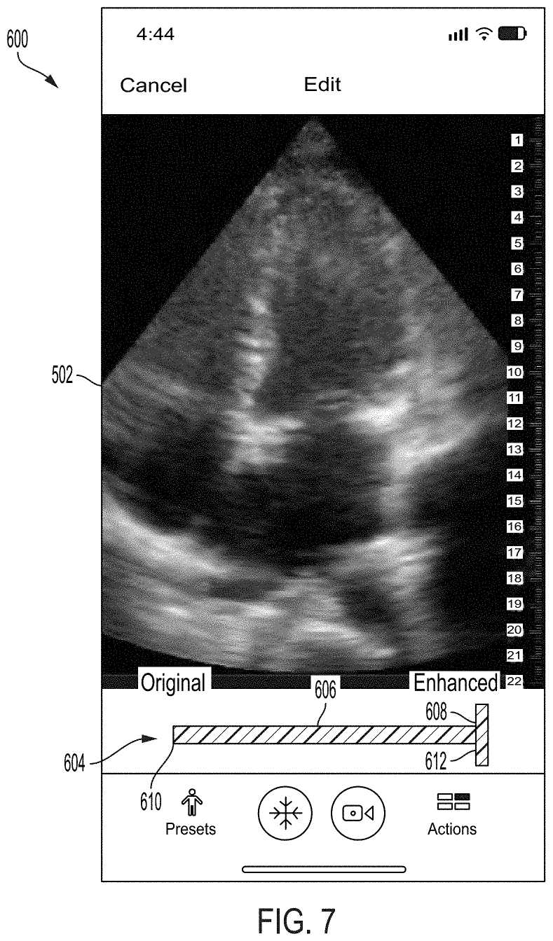

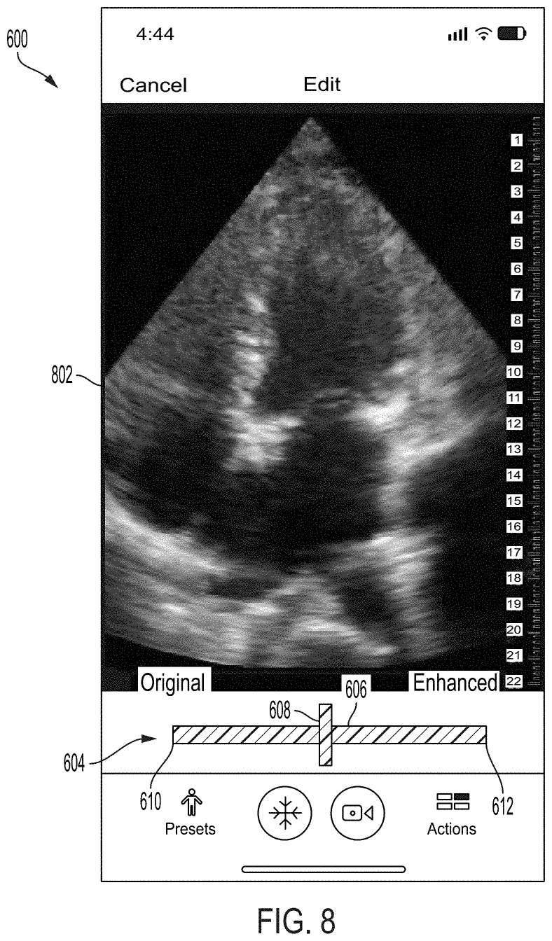

FIGS. 6-8 illustrate an example GUI 600. The GUI 600 may be an alternative to the GUI 500. The GUI 600 illustrates the ultrasound image 402 and an enhancement slider 604. The enhancement slider 604 includes a bar 606 and a slider 608. The bar 606 has a first end 610 and a second end 612. The first end 610 is marked by an “Original” label and the second end 612 is marked by an “Enhanced” label. A user may slide the slider 608 along the bar 606 (e.g., by touching the slider 608, dragging, and releasing on a touch-sensitive display screen). The enhancement slider 604 may enable a user to select a level of enhancement of the ultrasound image displayed by the processing device by sliding the slider 608 to a particular position along the bar 606. If the slider 608 is positioned at the first end 610, the processing device may display the original ultrasound image 402, as illustrated in the FIG. 6. If the slider 608 is positioned at the second end 612, the processing device may display the fully enhanced ultrasound image 502 (generated as described above), as illustrated in the FIG. 7. If the slider 608 is positioned between the first end 610 and the second end 612, the processing device may display a partially enhanced ultrasound image. If the slider 608 is positioned closer to the first end 610 of the bar 606, then the processing device may display in the GUI 600 an ultrasound image that is enhanced less than if the slider 608 is positioned closer to the second end 612 of the bar 606. The processing device may generate a partially enhanced ultrasound image by interpolating between the original ultrasound image 402 and the fully enhanced ultrasound image 502. In particular, consider that f is the distance of the slider 608 from the first end 610 of the bar 606 divided by the length of the bar 606 from the first end 610 to the second end 612 of the bar 606. In other words, the slider 608 is positioned a fraction f of the distance along the bar 606 from the first end 610. Further, consider that the value of a pixel at a particular location (x,y) in the original ultrasound image 402 is original(x,y) and the value of a pixel at the particular location (x,y) in the fully enhanced ultrasound image 502 (generated as described above) is enhanced(x,y). The processing device may display an ultrasound image where the value of each pixel is (1−f)(original(x,y))+f(enhanced(x,y)). For example, in FIG. 8, the slider 608 is positioned along the bar 606 halfway between the first end 610 and the second end 612, such that the processing device generates and displays an ultrasound image 802 where each pixel is the sum of half the value of the corresponding pixel in the ultrasound image 402 and half the value of the corresponding pixel in the ultrasound image 502. While the slider 604 may enable selection from a continuous range of enhancement levels, in some embodiments a slider may enable selection of discrete levels of enhancement.

FIG. 9 illustrates an example GUI 900. The GUI 900 may be an alternative to the GUI 500. The GUI 900 includes an ultrasound image 902, an enhancement region 904, and the original option 504. A user may move the enhancement region 904 across the ultrasound image 902 (e.g., by touching the enhancement region 904, dragging, and releasing on a touch-sensitive display screen). In some embodiments, a user may resize and/or reshape the enhancement region 904 (e.g., by performing a pinching gesture on a touch-sensitive display screen or by using controls on the GUI 900 that are not illustrated). The enhancement region 904 may enable a user to select a particular region of the ultrasound image 902 displayed by the processing device to enhance by moving the enhancement region 904 to that region. In particular, consider that the value of a pixel at a particular location (x,y) in the original ultrasound image 402 is original(x,y) and the value of a pixel at the particular location (x,y) in the fully enhanced ultrasound image 502 (generated as described above) is enhanced(x,y). Further, consider that mask is a matrix equal in size to the ultrasound images 402 and 502, where pixels in mask corresponding to the location of the enhancement region 904 are equal to 1 and other pixels are 0. Then the ultrasound image 902 may be equal to original (x,y)*(1−mask(x,y))+enhanced (x,y)*mask(x,y), where the multiplication operator indicates pixel-by-pixel multiplication.

In some embodiments, in response to receiving a selection of the original option 504, the processing device may display the GUI 400 (i.e., show the original ultrasound image 402 rather than the enhanced ultrasound image 502). If the ultrasound 402 was displayed in real-time, the processing device may cease to enhance subsequent ultrasound images collected by the ultrasound device in real-time. If the ultrasound image 402 was previously stored, the processing device may cease to enhance subsequent ultrasound images that are retrieved. If the ultrasound image 402 is displayed as part of a previously stored cine, the processing device may cease to enhance ultrasound images in the cine while displaying the cine.

In some embodiments (e.g., when the processing device is implementing the process 100C), the processing device may initially show a GUI that is the same as the GUIs 500, 600, or 900, but without the original option 504. The processing device may be performing image enhancement specific to a particular anatomical view or a particular set of anatomical views. If the processing device determines (e.g., using a statistical model) that the ultrasound image displayed in the GUI does not depict the particular anatomical view or one of the particular set of anatomical views specific to the image enhancement, the processing device may display the original option 504. In response to selection of the original option 504, the processing device may display the GUI 400, which may include an ultrasound image with no image enhancement performed.

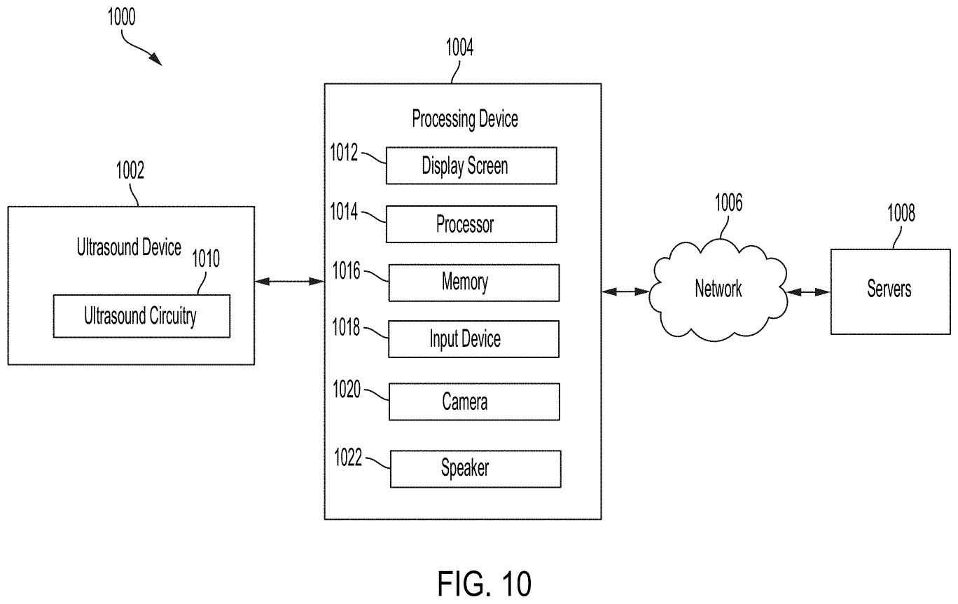

FIG. 10 illustrates a schematic block diagram of an example ultrasound system 1000 upon which various aspects of the technology described herein may be practiced. The ultrasound system 1000 includes an ultrasound device 1002, a processing device 1004, a network 1006, and one or more servers 1008. The processing device 1004 may be any of the processing devices described herein. The ultrasound device 1002 may be any of the ultrasound devices described herein.

The ultrasound device 1002 includes ultrasound circuitry 1010. The processing device 1004 includes a camera 1020, a display screen 1012, a processor 1014, a memory 1016, an input device 1018, and a speaker 1022. The processing device 1004 is in wired (e.g., through a lightning connector or a mini-USB connector) and/or wireless communication (e.g., using BLUETOOTH, ZIGBEE, and/or WiFi wireless protocols) with the ultrasound device 1002. The processing device 1004 is in wireless communication with the one or more servers 1008 over the network 1006.

The ultrasound device 1002 may be configured to generate ultrasound data that may be employed to generate an ultrasound image. The ultrasound device 1002 may be constructed in any of a variety of ways. In some embodiments, the ultrasound device 1002 includes a transmitter that transmits a signal to a transmit beamformer which in turn drives transducer elements within a transducer array to emit pulsed ultrasonic signals into a structure, such as a patient. The pulsed ultrasonic signals may be back-scattered from structures in the body, such as blood cells or muscular tissue, to produce echoes that return to the transducer elements. These echoes may then be converted into electrical signals by the transducer elements and the electrical signals are received by a receiver. The electrical signals representing the received echoes are sent to a receive beamformer that outputs ultrasound data. The ultrasound circuitry 1010 may be configured to generate the ultrasound data. The ultrasound circuitry 1010 may include one or more ultrasonic transducers monolithically integrated onto a single semiconductor die. The ultrasonic transducers may include, for example, one or more capacitive micromachined ultrasonic transducers (CMUTs), one or more CMOS (complementary metal-oxide-semiconductor) ultrasonic transducers (CUTs), one or more piezoelectric micromachined ultrasonic transducers (PMUTs), and/or one or more other suitable ultrasonic transducer cells. In some embodiments, the ultrasonic transducers may be formed on the same chip as other electronic components in the ultrasound circuitry 1010 (e.g., transmit circuitry, receive circuitry, control circuitry, power management circuitry, and processing circuitry) to form a monolithic ultrasound device. The ultrasound device 1002 may transmit ultrasound data and/or ultrasound images to the processing device 1004 over a wired (e.g., through a lightning connector or a mini-USB connector) and/or wireless (e.g., using BLUETOOTH, ZIGBEE, and/or WiFi wireless protocols) communication link.

Referring now to the processing device 1004, the processor 1014 may include specially-programmed and/or special-purpose hardware such as an application-specific integrated circuit (ASIC). For example, the processor 1014 may include one or more graphics processing units (GPUs) and/or one or more tensor processing units (TPUs). TPUs may be ASICs specifically designed for machine learning (e.g., deep learning). The TPUs may be employed, for example, to accelerate the inference phase of a neural network. The processing device 1004 may be configured to process the ultrasound data received from the ultrasound device 1002 to generate ultrasound images for display on the display screen 1012. The processing may be performed by, for example, the processor 1014. The processor 1014 may also be adapted to control the acquisition of ultrasound data with the ultrasound device 1002. The ultrasound data may be processed in real-time during a scanning session as the echo signals are received. In some embodiments, the displayed ultrasound image may be updated a rate of at least 5 Hz, at least 10 Hz, at least 20 Hz, at a rate between 5 and 60 Hz, at a rate of more than 20 Hz. For example, ultrasound data may be acquired even as images are being generated based on previously acquired data and while a live ultrasound image is being displayed. As additional ultrasound data is acquired, additional frames or images generated from more-recently acquired ultrasound data may be sequentially displayed. Additionally, or alternatively, the ultrasound data may be stored temporarily in a buffer during a scanning session and processed in less than real-time.

The processing device 1004 may be configured to perform certain of the processes (e.g., the processes 100A-100D) described herein using the processor 1014 (e.g., one or more computer hardware processors) and one or more articles of manufacture that include non-transitory computer-readable storage media such as the memory 1016. The processor 1014 may control writing data to and reading data from the memory 1016 in any suitable manner. To perform certain of the processes described herein, the processor 1014 may execute one or more processor-executable instructions stored in one or more non-transitory computer-readable storage media (e.g., the memory 1016), which may serve as non-transitory computer-readable storage media storing processor-executable instructions for execution by the processor 1014. The camera 1020 may be configured to detect light (e.g., visible light) to form an image. The camera 1020 may be on the same face of the processing device 1004 as the display screen 1012. The display screen 1012 may be configured to display images and/or videos, and may be, for example, a liquid crystal display (LCD), a plasma display, and/or an organic light emitting diode (OLED) display on the processing device 1004. The input device 1018 may include one or more devices capable of receiving input from a user and transmitting the input to the processor 1014. For example, the input device 1018 may include a keyboard, a mouse, a microphone, touch-enabled sensors on the display screen 1012, and/or a microphone. The display screen 1012, the input device 1018, the camera 1020, and the speaker 1022 may be communicatively coupled to the processor 1014 and/or under the control of the processor 1014.

It should be appreciated that the processing device 1004 may be implemented in any of a variety of ways. For example, the processing device 1004 may be implemented as a handheld device such as a mobile smartphone or a tablet. Thereby, a user of the ultrasound device 1002 may be able to operate the ultrasound device 1002 with one hand and hold the processing device 1004 with another hand. In other examples, the processing device 1004 may be implemented as a portable device that is not a handheld device, such as a laptop. In yet other examples, the processing device 1004 may be implemented as a stationary device such as a desktop computer. The processing device 1004 may be connected to the network 1006 over a wired connection (e.g., via an Ethernet cable) and/or a wireless connection (e.g., over a WiFi network). The processing device 1004 may thereby communicate with (e.g., transmit data to or receive data from) the one or more servers 1008 over the network 1006. For example, a party may provide from the server 1008 to the processing device 1004 processor-executable instructions for storing in one or more non-transitory computer-readable storage media (e.g., the memory 1016) which, when executed, may cause the processing device 1004 to perform certain of the processes (e.g., the processes 100A-100D) described herein.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

As used herein, reference to a numerical value being between two endpoints should be understood to encompass the situation in which the numerical value can assume either of the endpoints. For example, stating that a characteristic has a value between A and B, or between approximately A and B, should be understood to mean that the indicated range is inclusive of the endpoints A and B unless otherwise noted.

The terms “approximately” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having described above several aspects of at least one embodiment, it is to be appreciated various alterations, modifications, and improvements will readily occur to those skilled in the art. Such alterations, modifications, and improvements are intended to be object of this disclosure. Accordingly, the foregoing description and drawings are by way of example only.

The present disclosure also includes the following numbered clauses:

B1. A method, comprising: receiving ultrasound data; automatically determining, with a processing device, that the ultrasound data depicts an anatomical view or one of a set of anatomical views; based on automatically determining that the ultrasound data depicts the anatomical view or one of the set of anatomical views, enabling an option to perform ultrasound data enhancement specific to the anatomical view or the set of anatomical views; receiving a selection of the option; and based on receiving the selection of the option, enhancing the ultrasound data, a portion thereof, and/or subsequently-collected ultrasound data using the ultrasound data enhancement specific to the anatomical view or the set of anatomical views.

B2. The method of clause B1, wherein receiving the ultrasound data comprises receiving the ultrasound data from an ultrasound device in real-time as the ultrasound data is collected or generated by the ultrasound device.

B3. The method of clause B1, wherein receiving the ultrasound data comprises retrieving ultrasound data that has been previously stored.

B4. The method of clause B1, wherein automatically determining that the ultrasound data depicts the anatomical view or one of the set of anatomical views comprises using one or more statistical models and/or deep learning techniques.

B5. The method of clause B1, wherein the anatomical view comprises one of an apical two chamber view of a heart, an apical four chamber view of the heart, a parasternal long axis view of the heart, and a parasternal short axis view of the heart.

B6. The method of clause B1, wherein the set of anatomical views comprises an apical two chamber view of a heart, an apical four chamber view of the heart, a parasternal long axis view of the heart, and a parasternal short axis view of the heart.

B7. The method of clause B1, wherein the anatomical view comprises a three-dimensional view of a fetus.

B8. The method of clause B1, wherein enabling the option comprises enabling a user to select the option.

B9. The method of clause B1, wherein enabling the option comprises enabling an action to be performed upon selection of the option.

B10. The method of clause B1, wherein enabling the option comprises displaying an option that was not previously displayed by the processing device.

B11. The method of clause B1, wherein enabling the option comprises changing a manner of display of the option.

B12. The method of clause B1, wherein enhancing the ultrasound data, the portion thereof, and/or the subsequently-collected ultrasound data comprises using a statistical model trained to convert ultrasound data from an initial domain to a final domain, where the initial domain includes low-quality ultrasound data and the final domain includes high-quality ultrasound data.

B13. The method of clause B12, wherein the statistical model is specifically trained on ultrasound data depicting the anatomical view or the set of anatomical views.

B14. The method of clause B1, wherein enhancing the ultrasound data, the portion thereof, and/or the subsequently-collected ultrasound data comprises enhancing the ultrasound data using a user-selectable degree of enhancement.

B15. The method of clause B1, wherein enhancing the ultrasound data, the portion thereof, and/or the subsequently-collected ultrasound data comprises enhancing portions of the ultrasound data non-uniformly.

B16. The method of clause B15, wherein enhancing the portions of the ultrasound data non-uniformly comprises enhancing first portions of an ultrasound image more than second portions of the ultrasound image, the first portions being closer to a user-selected point than the second portions.

B17. The method of clause B1, wherein enhancing the ultrasound data, the portion thereof, and/or the subsequently-collected ultrasound data comprises only enhancing a portion of the ultrasound data.

B18. The method of clause B17, wherein only enhancing the portion of the ultrasound data comprises only enhancing a portion of an ultrasound image within a user-selectable region.

C1. At least one non-transitory computer-readable storage medium storing processor-executable instructions that, when executed by at least one processor on a processing device in operative communication with an ultrasound device, cause the at least one processor to perform a method as set out in at least one of clauses B1 to B18.

D1. An apparatus, comprising a processing device configured to perform a method as set out in at least one of clauses B1 to B18.

E1. A method, comprising: receiving ultrasound data; automatically determining, with a processing device, that the ultrasound data depicts an anatomical view or one of a set of anatomical views; based on automatically determining that the ultrasound data depicts the anatomical view or one of the set of anatomical views, enabling an option to perform ultrasound data enhancement specific to the anatomical view or the set of anatomical views; receiving a selection of the option; and based on receiving the selection of the option, enhancing the ultrasound data, a portion thereof, and/or subsequently-collected ultrasound data using the ultrasound data enhancement specific to the anatomical view or the set of anatomical views.

E2. The method of clause E1, wherein receiving the ultrasound data comprises receiving the ultrasound data from an ultrasound device in real-time as the ultrasound data is collected or generated by the ultrasound device.

E3. The method of clause E1, wherein receiving the ultrasound data comprises retrieving ultrasound data that has been previously stored.

E4. The method of clause E1, wherein automatically determining that the ultrasound data depicts the anatomical view or one of the set of anatomical views comprises using one or more statistical models and/or deep learning techniques.

E5. The method of clause E1, wherein the anatomical view comprises one of an apical two chamber view of a heart, an apical four chamber view of the heart, a parasternal long axis view of the heart, and a parasternal short axis view of the heart.

E6. The method of clause E1, wherein the set of anatomical views comprises an apical two chamber view of a heart, an apical four chamber view of the heart, a parasternal long axis view of the heart, and a parasternal short axis view of the heart.

E7. The method of clause E1, wherein the anatomical view comprises a three-dimensional view of a fetus.

E12. The method of clause E1, wherein enhancing the ultrasound data, the portion thereof, and/or the subsequently-collected ultrasound data comprises using a statistical model trained to convert ultrasound data from an initial domain to a final domain, where the initial domain includes low-quality ultrasound data and the final domain includes high-quality ultrasound data.

E13. The method of clause E12, wherein the statistical model is specifically trained on ultrasound data depicting the anatomical view or the set of anatomical views.

E14. The method of clause E1, wherein enhancing the ultrasound data, the portion thereof, and/or the subsequently-collected ultrasound data comprises enhancing the ultrasound data using a user-selectable degree of enhancement.

E15. The method of clause E1, wherein enhancing the ultrasound data, the portion thereof, and/or the subsequently-collected ultrasound data comprises enhancing portions of the ultrasound data non-uniformly.

E16. The method of clause E15, wherein enhancing the portions of the ultrasound data non-uniformly comprises enhancing first portions of an ultrasound image more than second portions of the ultrasound image, the first portions being closer to a user-selected point than the second portions.

E17. The method of clause E1, wherein enhancing the ultrasound data, the portion thereof, and/or the subsequently-collected ultrasound data comprises only enhancing a portion of the ultrasound data.

E18. The method of clause E17, wherein only enhancing the portion of the ultrasound data comprises only enhancing a portion of an ultrasound image within a user-selectable region.

F1. At least one non-transitory computer-readable storage medium storing processor-executable instructions that, when executed by at least one processor on a processing device in operative communication with an ultrasound device, cause the at least one processor to perform a method as set out in at least one of clauses E1 to E18.

G1. An apparatus, comprising a processing device configured to perform a method as set out in at least one of clauses E1 to E18.

H1. A method, comprising: receiving ultrasound data; automatically determining, with a processing device, that the ultrasound data does not depict an anatomical view or one of a set of anatomical views specific to ultrasound data enhancement being performed; based on automatically determining that the ultrasound data does not depict the anatomical view or one of the set of anatomical views, enabling an option to cease to perform the ultrasound data enhancement specific to the anatomical view or the set of anatomical views; receiving a selection of the option; and based on receiving the selection of the option, ceasing to enhance the ultrasound data, a portion thereof, and/or subsequently-collected ultrasound data using the ultrasound data enhancement specific to the anatomical view or the set of anatomical views.

H2. The method of clause H1, wherein receiving the ultrasound data comprises receiving the ultrasound data from an ultrasound device in real-time as the ultrasound data is collected or generated by the ultrasound device.

H3. The method of clause H1, wherein receiving the ultrasound data comprises retrieving ultrasound data that has been previously stored.

H4. The method of clause H1, wherein automatically determining that the ultrasound data does not depict the anatomical view or one of the set of anatomical views comprises using one or more statistical models and/or deep learning techniques.

H5. The method of clause H1, wherein the anatomical view comprises one of an apical two chamber view of a heart, an apical four chamber view of the heart, a parasternal long axis view of the heart, and a parasternal short axis view of the heart.

H6. The method of clause H1, wherein the set of anatomical views comprises an apical two chamber view of a heart, an apical four chamber view of the heart, a parasternal long axis view of the heart, and a parasternal short axis view of the heart.

H7. The method of clause H1, wherein the anatomical view comprises a three-dimensional view of a fetus.

H8. The method of clause H1, wherein enabling the option comprises enabling a user to select the option.

H9. The method of clause H1, wherein enabling the option comprises enabling an action to be performed upon selection of the option.

H10. The method of clause H1, wherein enabling the option comprises displaying an option that was not previously displayed by the processing device.

H11. The method of clause H1, wherein enabling the option comprises changing a manner of display of the option.

Il. At least one non-transitory computer-readable storage medium storing processor-executable instructions that, when executed by at least one processor on a processing device in operative communication with an ultrasound device, cause the at least one processor to perform a method as set out in at least one of clauses H1 to H11.

J1. An apparatus, comprising a processing device configured to perform a method as set out in at least one of clauses H1 to H11.

K1. A method, comprising: receiving ultrasound data; automatically determining, with a processing device, that the ultrasound data does not depict an anatomical view or one of a set of anatomical views specific to ultrasound data enhancement being performed; and based on automatically determining that the ultrasound data does not depict the anatomical view or one of the set of anatomical views, ceasing to enhance the ultrasound data, a portion thereof, and/or subsequently-collected ultrasound data using the ultrasound data enhancement specific to the anatomical view or the set of anatomical views.

K2. The method of clause K1, wherein receiving the ultrasound data comprises receiving the ultrasound data from an ultrasound device in real-time as the ultrasound data is collected or generated by the ultrasound device.

K3. The method of clause K1, wherein receiving the ultrasound data comprises retrieving ultrasound data that has been previously stored.

K4. The method of clause K1, wherein automatically determining that the ultrasound data does not depict the anatomical view or one of the set of anatomical views comprises using one or more statistical models and/or deep learning techniques.

K5. The method of clause K1, wherein the anatomical view comprises one of an apical two chamber view of a heart, an apical four chamber view of the heart, a parasternal long axis view of the heart, and a parasternal short axis view of the heart.

K6. The method of clause K1, wherein the set of anatomical views comprises an apical two chamber view of a heart, an apical four chamber view of the heart, a parasternal long axis view of the heart, and a parasternal short axis view of the heart.

K7. The method of clause K1, wherein the anatomical view comprises a three-dimensional view of a fetus.

L1. At least one non-transitory computer-readable storage medium storing processor-executable instructions that, when executed by at least one processor on a processing device in operative communication with an ultrasound device, cause the at least one processor to perform a method as set out in at least one of clauses K1 to K7.

M1. An apparatus, comprising a processing device configured to perform a method as set out in at least one of clauses K1 to K7.

What is claimed is:

1. An apparatus, comprising:
a processing device configured to:
receive ultrasound data;
automatically determine that the ultrasound data depicts an anatomical view or one of a set of anatomical views;
based on automatically determining that the ultrasound data depicts the anatomical view or one of the set of anatomical views, enable an option to perform ultrasound data enhancement specific to the anatomical view or the set of anatomical views;
receive a selection of the option; and
based on receiving the selection of the option, enhance the ultrasound data, a portion thereof, and/or subsequently-collected ultrasound data using the ultrasound data enhancement specific to the anatomical view or the set of anatomical views.

2. The apparatus of claim 1, wherein the processing device is in operative communication with an ultrasound device, and the processing device is configured, when receiving the ultrasound data, to receive the ultrasound data from the ultrasound device in real-time as the ultrasound data is collected or generated by the ultrasound device.

3. The apparatus of claim 1, wherein the processing device is configured, when receiving the ultrasound data, to retrieve ultrasound data that has been previously stored.

4. The apparatus of claim 1, wherein the processing device is configured, when automatically determining that the ultrasound data depicts the anatomical view or one of the set of anatomical views, to use one or more statistical models and/or deep learning techniques.

5. The apparatus of claim 1, wherein the anatomical view comprises one of an apical two chamber view of a heart, an apical four chamber view of the heart, a parasternal long axis view of the heart, and a parasternal short axis view of the heart.

6. The apparatus of claim 1, wherein the set of anatomical views comprises an apical two chamber view of a heart, an apical four chamber view of the heart, a parasternal long axis view of the heart, and a parasternal short axis view of the heart.

7. The apparatus of claim 1, wherein the anatomical view comprises a three-dimensional view of a fetus.

8. The apparatus of claim 1, wherein the processing device is configured, when enabling the option, to enable a user to select the option.

9. The apparatus of claim 1, wherein the processing device is configured, when enabling the option, to enable an action to be performed upon selection of the option.

10. The apparatus of claim 1, wherein the processing device is configured, when enabling the option, to display an option that was not previously displayed by the processing device.

11. The apparatus of claim 1, wherein the processing device is configured, when enabling the option, to change a manner of display of the option.

12. The apparatus of claim 1, wherein the processing device is configured, when enhancing the ultrasound data, the portion thereof, and/or the subsequently-collected ultrasound data, to use a statistical model trained to convert ultrasound data from an initial domain to a final domain, where the initial domain includes low-quality ultrasound data and the final domain includes high-quality ultrasound data.

13. The apparatus of claim 12, wherein the statistical model is specifically trained on ultrasound data depicting the anatomical view or the set of anatomical views.

14. The apparatus of claim 1, wherein the processing device is configured, when enhancing the ultrasound data, the portion thereof, and/or the subsequently-collected ultrasound data, to enhance the ultrasound data using a user-selectable degree of enhancement.

15. The apparatus of claim 1, wherein the processing device is configured, when enhancing the ultrasound data, the portion thereof, and/or the subsequently-collected ultrasound data, to enhance portions of the ultrasound data non-uniformly.

16. The apparatus of claim 15, wherein the processing device is configured, when enhancing the portions of the ultrasound data non-uniformly, to enhance first portions of an ultrasound image more than second portions of the ultrasound image, the first portions being closer to a user-selected point than the second portions.

17. The apparatus of claim 1, wherein the processing device is configured, when enhancing the ultrasound data, the portion thereof, and/or the subsequently-collected ultrasound data, to only enhance a portion of the ultrasound data.

18. The apparatus of claim 17, wherein the processing device is configured, when only enhancing the portion of the ultrasound data, to only enhance a portion of an ultrasound image within a user-selectable region.

19. A method, comprising:
receiving ultrasound data;
automatically determining, with a processing device, that the ultrasound data depicts an anatomical view or one of a set of anatomical views;
based on automatically determining that the ultrasound data depicts the anatomical view or one of the set of anatomical views, enabling an option to perform ultrasound data enhancement specific to the anatomical view or the set of anatomical views;
receiving a selection of the option; and
based on receiving the selection of the option, enhancing the ultrasound data, a portion thereof, and/or subsequently-collected ultrasound data using the ultrasound data enhancement specific to the anatomical view or the set of anatomical views.

20. At least one non-transitory computer-readable storage medium storing processor-executable instructions that, when executed by at least one processor on a processing device in operative communication with an ultrasound device, cause the at least one processor to:
receive ultrasound data;
automatically determine that the ultrasound data depicts an anatomical view or one of a set of anatomical views;
based on automatically determining that the ultrasound data depicts the anatomical view or one of the set of anatomical views, enable an option to perform ultrasound data enhancement specific to the anatomical view or the set of anatomical views;
receive a selection of the option; and
based on receiving the selection of the option, enhance the ultrasound data, a portion thereof, and/or subsequently-collected ultrasound data using the ultrasound data enhancement specific to the anatomical view or the set of anatomical views.
